(19) United States
(12) Patent Application Publication
(10) Pub. No.: US 2022/0202221 A1
(43) Pub. Date: Jun. 30, 2022

(54) GLOVE DISPENSER AND GLOVE OPENING MECHANISM THEREFOR

(71) Applicant: iNitrile INC, Houston, TX (US)

(72) Inventors: Omri Shafran, Houston, TX (US); Dimitri Menin, Houston, TX (US); Ricardo Espejel Garcia, San Pedro Cholula (MX); Alejandro Gutierrez Prieto, Puebla (MX); Juan Jose Macias Luna, Puebla (MX); Fernando Ramirez Prieto, Puebla (MX); Raul Arroyo Martinez Fabre, Queretaro (MX); Hector Jonas Delgadillo Castell, Colonia Nueva Galicia residencial (MX); Slavomir Soltys, Bardejov (SK)

(21) Appl. No.: 17/134,007

(22) Filed: Dec. 24, 2020

(51) Int. Cl. A47G 25/90

(52) U.S. Cl. CPC A47G 25/904

(57) ABSTRACT

A glove dispenser for dispensing a glove. The dispenser includes a cabinet with a hand-insertion port; a glove storage container housed within the cabinet and includes at least one glove bin configured to store a plurality of gloves. The dispenser also includes a glove picking and positioning mechanism configured to remove one glove from one of the at least one glove storage bins and to fix the glove in a position accessible via the hand-insertion port; and a glove-cuff opening device.

### TECHNOLOGICAL FIELD

The present invention is in the field of dispensers, in particular dispensers for providing gloves, for example hygienic gloves such as those used in the health care industry, laboratories and the like.

### BACKGROUND

Sanitary or hygienic gloves are commonly used in the medical industry (e.g. by doctors, nurses, dentists and the like), as well as in laboratory settings, the food industry and so on. Presently, contamination by viruses such as COVID-19, are a particular concern.

Hygienic gloves are typically made of a rubbery plastic/polymer such as latex, which can be difficult and/or slow to put on; and donning the gloves manually can cause contamination thereof.

U.S. Pat. No. 9,931,174 (Machado et al., 2018 Apr. 3) discloses a glove dispenser where gloves are dispensed from a roll of sequential gloves. An electro-mechanical feed mechanism rotates the glove roll. A proximity sensor detects a user's hand and activates rotation of the glove roll.

US patent application 2016/152403 (Ray, 2016 Jun. 2) discloses a sanitary glove dispensing apparatus where glove pairs overlap in a pre-determined pattern, by both cuff ends, so finger ends of the protective gloves are not contaminated during dispensing.

The teachings of the aforementioned publications are incorporated by reference as if fully set forth herein.

### GENERAL DESCRIPTION

The present invention relates to a dispensing machine and method for facilitating donning a glove on a user. The machine or dispenser is configured to position and prepare a glove for the convenient insertion therein of the user's hand, into the glove. However, it should be understood that the dispenser can, mutatis mutandis, be suited to dressing of a sock or galosh-type item, or the like, on a user's foot; and/or potentially other implementations, for example, facilitating the dressing of a finger cot.

According to one aspect of the present invention there is provided a dispenser, which is particularly suited to position and dress a glove, or sock, or the like, on an extremity.

The dispenser includes a cabinet with a hand-insertion port; a glove storage container housed within the cabinet and including at least one glove bin configured to store a plurality of gloves; a glove picking and positioning mechanism configured to remove one glove from one of the at least one glove storage bins and to fix the glove in a position accessible via the hand-insertion port; and a glove-cuff opening device.

In some embodiments, the glove picking and positioning mechanism includes a glove picking arm with a glove-picking element attached to its free end.

In some embodiments, the glove-picking element has at least one glove-picking vacuum openings operably connected to a glove-picking vacuum pump.

In some embodiments, the glove picking arm is connected to one or more picking-arm movement rods upon which the picking arm can be moved back and forth.

In some embodiments, each glove bin is configured so that the gloves therein are arranged in a vertically stacked arrangement.

In some embodiments, there is a plurality of storage bins. In some embodiments, the storage bins are arranged radially in the glove storage container in an equidistant petal-like arrangement.

In some embodiments, the glove storage container is configured as a rotatable carousel. In some embodiments, the glove storage container is associated with a storage bin rotation mechanism configured to rotate the glove storage container.

In some embodiments, the dispenser further includes a cuff opening device configured to open the cuff of the glove and includes a cuff-opening suction plate operably connected to and moveable by a suction-plate movement motor and operably connected to a suction-plate vacuum pump.

In some embodiments, the dispenser further includes a cuff-stretching and fixing device configured to stretch the cuff of the positioned glove.

In some embodiments, the cuff stretching and fixing device is configured to stretch the glove's cuff adjacent to the inner periphery of a hand-insertion gateway frame.

In some embodiments, the dispenser further includes a gateway frame upon which the glove's cuff is fixable by the cuff-stretching and fixing device.

In some embodiments, the cuff-stretching and fixing device includes cuff-stretching claw arms cuff-stretching pins at free ends of the claw arms; and cuff-stretching claw motors configured to move claw arms so as to spread and converge the cuff-stretching pins.

In some embodiments, the dispenser further includes a glove request sensor or instruction screen configured to allow a user to request one glove of the plurality of gloves.

In some embodiments, the dispenser further includes an instruction screen configured to allow a user to indicate a desired glove size.

In some embodiments, the dispenser further includes cabinet air-extraction fan for inflating the glove.

In some embodiments, each glove bin has associated therewith a linear glove-stack pushing device configured to raise a stack of gloves located within each glove bin.

In some embodiments, the dispenser further includes a hand sanitizer compartment for housing a hand sanitizer, the dispensing of the sanitizer being actuatable via a sanitizer dispensing sensor.

In accordance with another aspect of the present invention, there is provided a glove opening mechanism for a glove having a cuff. The glove opening mechanism includes a glove picking arm with a glove-picking element attached to its free end, the glove-picking element; a glove-picking vacuum pump operably attached to the glove-picking element; and a cuff-opening suction plate and operably connected to a suction-plate vacuum pump.

In some embodiments, the glove opening mechanism further includes a plurality of glove opening pins configured to enter the glove's cuff and a glove-cuff opening device configured to spread the pins within the cuff.

In some embodiments, the glove-cuff opening device includes cuff-stretching claw arms; cuff-stretching pins at free ends of the claw arms; and cuff-stretching claw motors operably connected to the claw arms and configured to move claw arms so as to spread and converge the plurality of cuff-stretching pins.

In some embodiments, the mechanism of further includes a cabinet air-extraction fan configured to lower the pressure within the cabinet for inflating the glove.

The term “glove” will be used herein to include any suitable hand, foot, finger covering, or the like, including but not limited to a glove, a sock, a finger cot and the like. The term “hand” will be used herein to include any suitable body extremity for being donned by a glove (as defined herein), typically a hand, a foot and a finger or a toe.

In preferred implementations, the glove opening mechanism is configured to open the glove in a manner suitable for a user's hand to be inserted with little or no friction. In some preferred implementations, the glove opening mechanism is configured to open the glove to a size greater than that of the user's hand.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a front perspective view of a glove dispenser in accordance with exemplary embodiments of the invention.

FIG. 2 is a rear perspective view of the glove dispenser.

FIGS. 3-5 are, respectively, two top perspective views, a bottom perspective view of a glove storage container of the glove dispenser, in accordance with embodiments of the invention.

FIGS. 6-8 are perspective views illustrating a glove picking and positioning mechanism of the dispenser, in accordance with embodiments of the invention.

FIGS. 9-13 are perspective views depicting a glove opening mechanism of the dispenser, in accordance with embodiments of the invention.

The following detailed description of embodiments of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION OF EMBODIMENTS

Illustrative embodiments of the invention are described below. In the interest of clarity, not all features/components of an actual implementation are necessarily described.

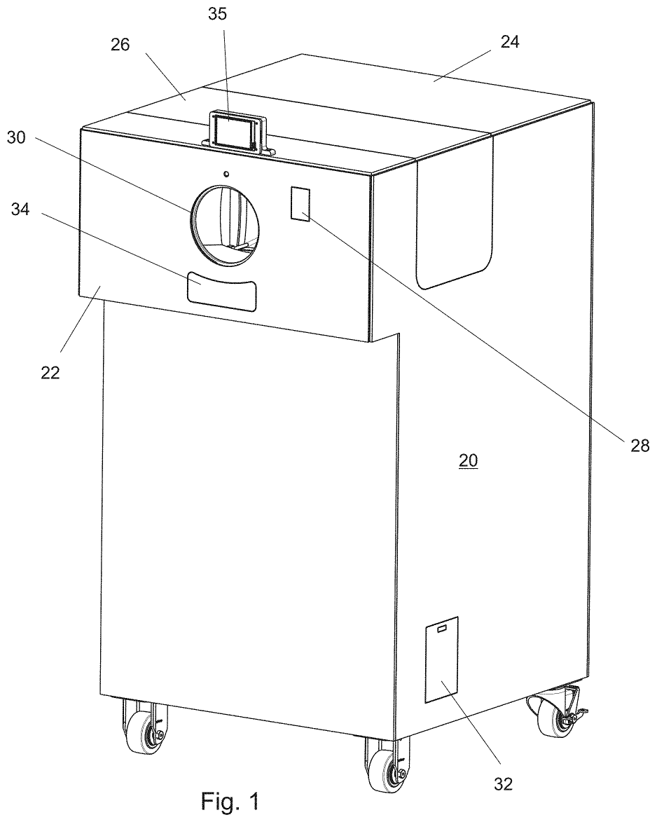

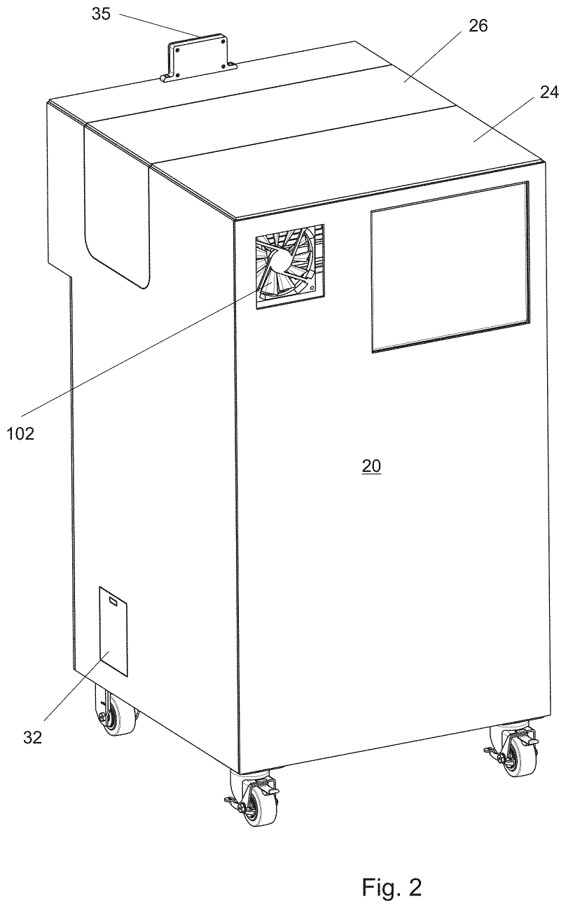

FIG. 1 and FIG. 2 show a glove dispenser in accordance with embodiments of the present invention. The glove dispenser includes a housing or cabinet 20 having a front cover 22 and a top cover 24, which may be openable to allow loading of gloves and may have a transparent window 26. At front cover 22 is a glove dispensing sensor 28 for the user to indicate their desire to have a glove provided, and which may also be used to choose the glove size. Front cover 22 may alternatively or additionally be openable for loading gloves and includes a hand-insertion port 30. Cabinet 20 may also include a sanitizer compartment 32 for housing a sanitizer that can be actuated via a sanitizer dispensing sensor 34. The dispenser may also include an instruction screen 35 for choosing a desired glove zo size and to indicate the inventory situation of gloves in the dispenser.

The dispenser may also include means for identifying the individual requesting a glove, for example via a badge swipe element, finger/palm print recognition, etc., and a suitable CPU. Such means can also be used for glove inventory control and ordering of an additional glove supply, as well as sanitizer inventory control and ordering sanitizer.

Described in more detail herein-below, the dispenser may also include a glove-size selection mechanism for receiving user glove-selection instructions and positioning a chosen glove size for retrieval, for example choosing a large, medium or small glove. Sensor 28 may be a component of glove-size selection mechanism.

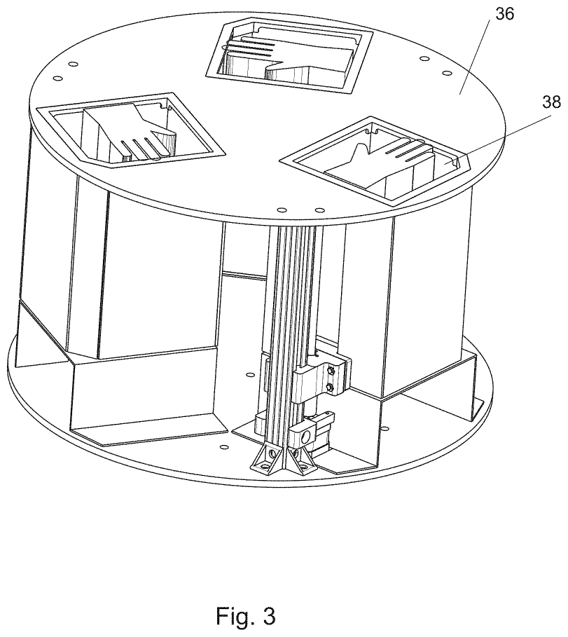

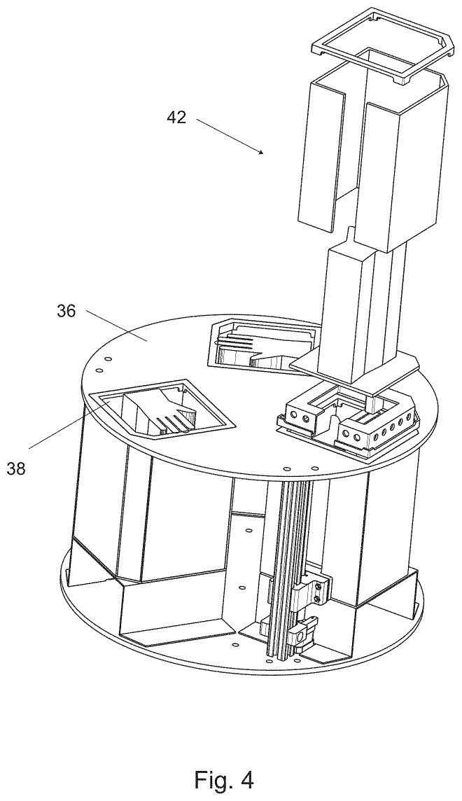

FIGS. 3-5 show a glove storage container/carousel 36, which has at least one glove-storage bin 38 (three bins are illustrated), each bin being configured to store a plurality of stacked gloves. The gloves may be supplied and loaded using a glove-supply sleeve 42 (FIG. 4), which may remain within each bin 38. Glove-storage bins 38 may be elongated vertical bins for storing stacked gloves. The three bins 38 illustrated, are arranged radially in an equidistant petal-like arrangement and glove storage container/carousel 36 is cylindrical and configured like a carousel.

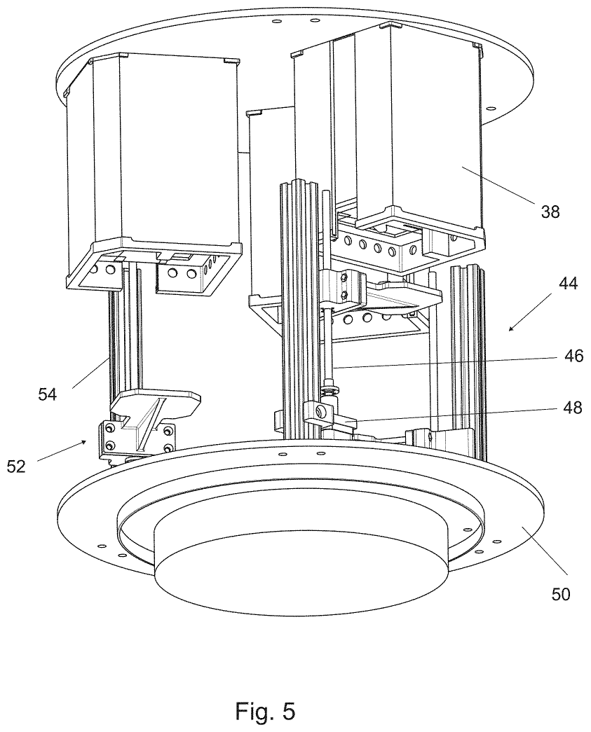

FIG. 5 shows a storage bin rotation mechanism 44 for rotating glove storage container/carousel 36. A carousel rotation plate 50 may be disposed under carousel 36 to facilitate the rotation of the carousel in order to position a desired glove-storage bin 38 in a location for being picked, via one or more pivot motors 48, such as stepper motors. Carousel 36 may be positioned on a forwardly extendable drawer (not shown) for glove loading via front cover 22.

Carousel 36 includes, or has associated therewith, a linear glove-stack pushing device 52 for raising the stack of gloves whereby the top glove will be reasonably level with the top of carousel 36 so as to be conveniently pickable. For such purpose, glove-stack pushing device 52 may have a glove-stack pushing rod 54 configured to suitably correspond in area/shape to the gloves in the glove stack.

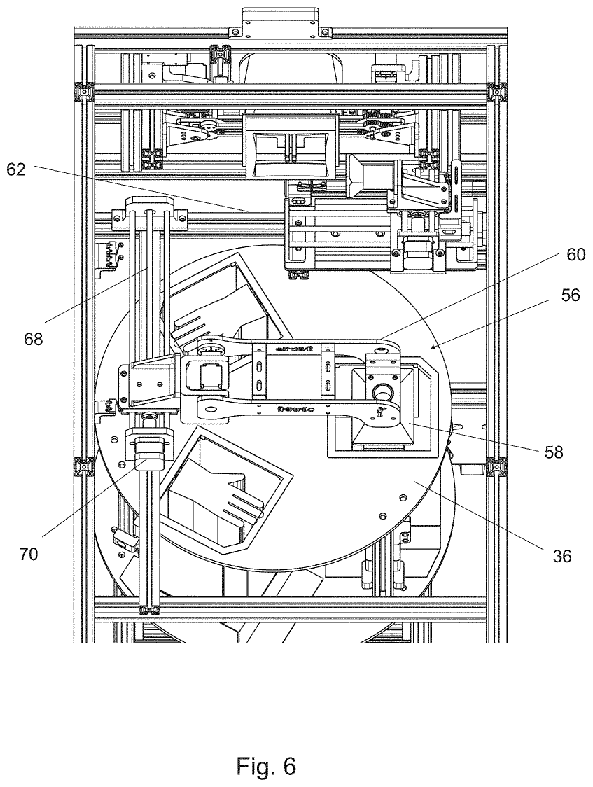

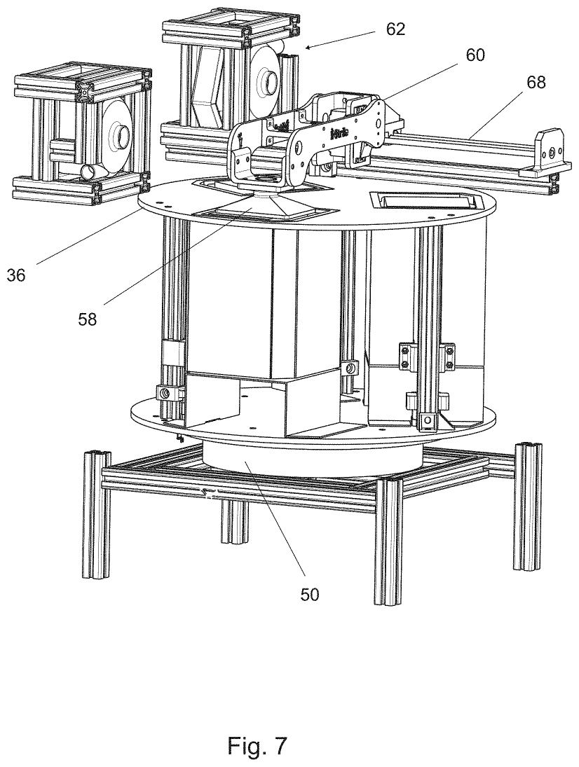

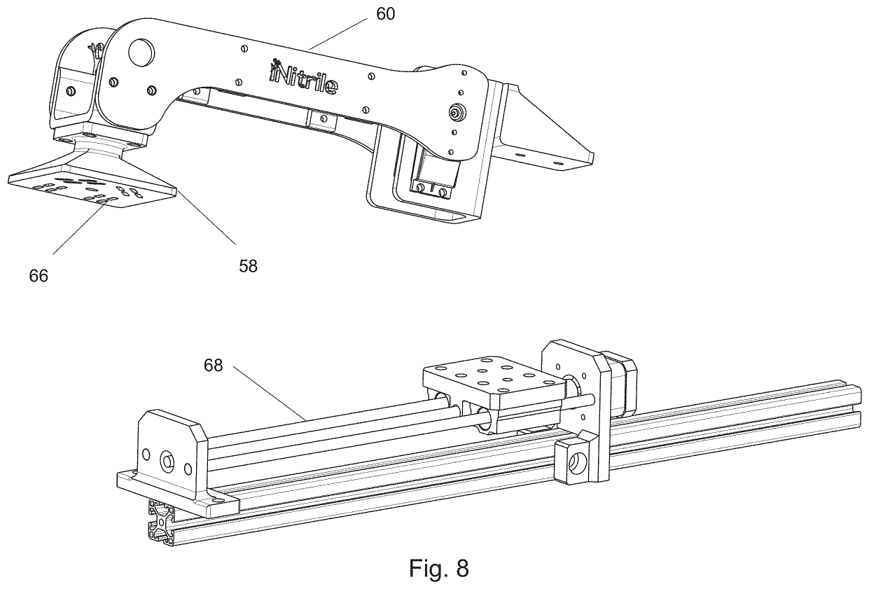

FIGS. 6-8 show a glove picking and positioning mechanism 56 of the dispenser. Glove picking and positioning mechanism 56 is designed to pick up a glove from a suitably positioned glove stack, and to position the glove adjacent hand-insertion port 30. For such purpose, glove picking and positioning mechanism 56 includes a glove-picking element 58 attached to a free end of a glove picking arm 60, such as the illustrated L-shaped arm. Glove-picking element 58 is operably connected to a glove-picking vacuum pump 62 (FIG. 7). Glove-picking element 58 has at least one and typically a plurality of glove-picking vacuum openings 66 (FIG. 8) on its lower side, for interfacing with the chosen glove.

Glove picking arm 60 is connected to one or more picking-arm movement rods 68 upon which the picking arm can be slid/moved back and forth via a stepper motor 70 (FIG. 6). Glove picking arm 60 is also attached to an arm lifting motor (not shown; e.g. a servo motor) configured to pivot the arm upward and downward, i.e. to lift and lower glove-picking element 58.

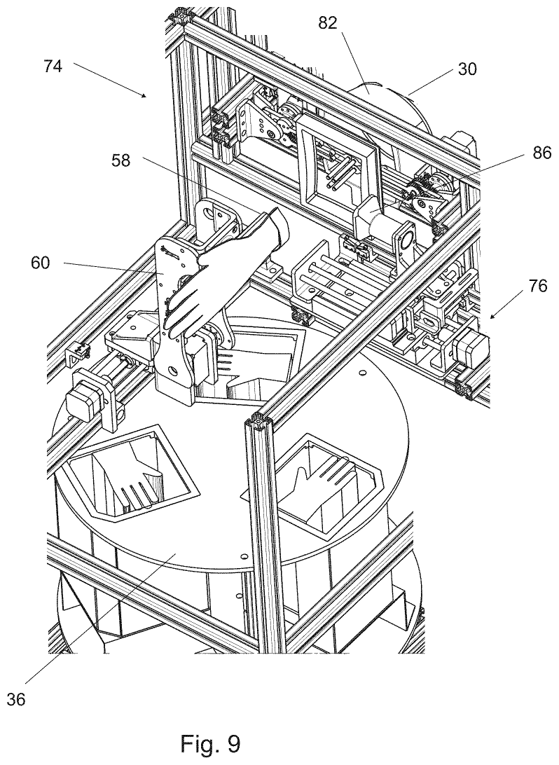

In FIG. 6, stepper motor 70 has positioned glove picking arm 60 so that the arm is parallel to the glove to be picked and the arm 60 is lowered so that glove-picking element 58 interfaces with the top glove in one of bins 38 after carousel 36 positions the chosen bin to the proper position for being picked up. Glove-picking vacuum pump 62 is activated to draw suction through glove-picking vacuum openings 66 of glove-picking element 58 whereby the glove adheres to element 58. Glove picking arm 60 then raises the glove and the arm is moved parallel to picking-arm movement rods 68 toward hand-insertion port 30 (FIG. 9). Glove picking arm 60 is then lowered , or otherwise positioned, to place the glove opposite hand-insertion port 30.

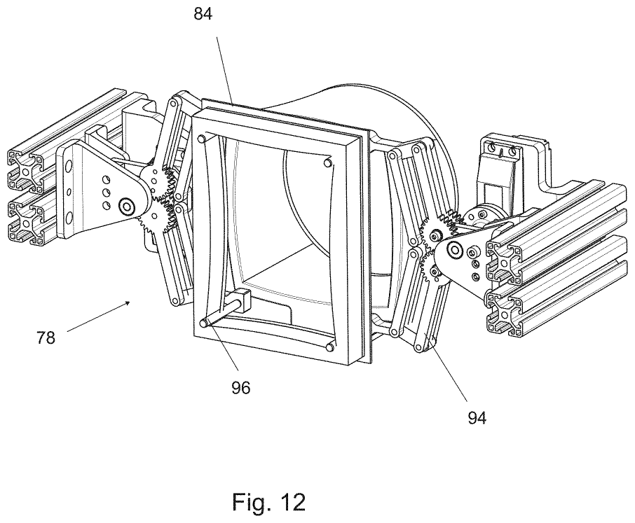

FIGS. 9-13 show various views and portions of a glove opening mechanism 74 of the dispenser, configured to open the glove into a ready state for 20 donning. The opening is performed in a few stages, via (a) a cuff opening device 76; (b) a cuff-stretching and fixing device 78; and (c) a glove inflation device 80.

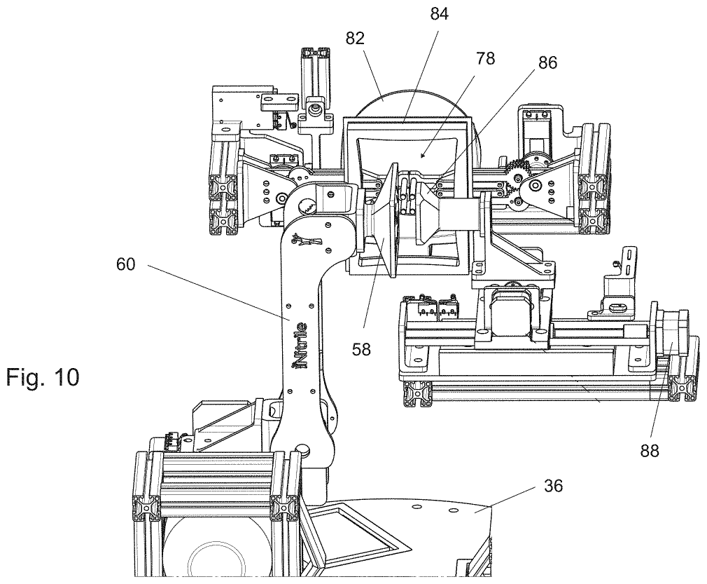

FIG. 10 shows glove opening mechanism's cuff opening device 76, which is activated when the glove is located opposite a hand-insertion gateway 82 of hand-insertion port 30, still being held by glove-picking element 58. In front of hand-insertion gateway 82 is a gateway frame 84 upon which the glove's cuff is fixable prior to donning of the glove. Cuff opening device 76 includes a cuff-opening suction plate 86 operably connected to and moveable by a suction-plate movement motor 88 and operably connected to a suction-plate vacuum pump 90 (FIG. 7).

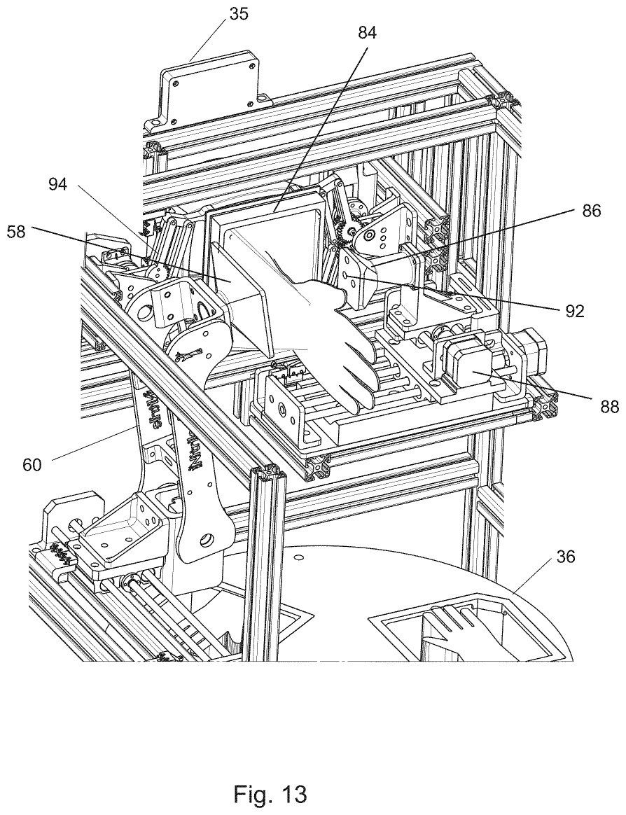

When cuff opening device 76 is activated, suction-plate movement motor 88 moves suction plate 86 forward to a position on an opposite side of glove-picking element 58 to interface with the glove, and suction-plate vacuum pump 90 is activated to draw air into plate vacuum apertures 92 (FIG. 13). Suction-plate movement motor 88 then moves suction plate 86 backward to partially open the cuff. Once the glove cuff is partially opened by picking element 58 and suction plate 86, motors 70 and 88 are activated to move forward in parallel to deliver the glove forward so that cuff-stretching pins 96 are introduced to the partially open glove. Then pins 96 are spread apart (e.g. approximately 30%) to hold the glove by its cuff. At this point glove-picking vacuum pump 62 and suction-plate vacuum pump 90 are deactivated, and glove picking arm 60 and suction plate 86 are retracted to their respective home positions.

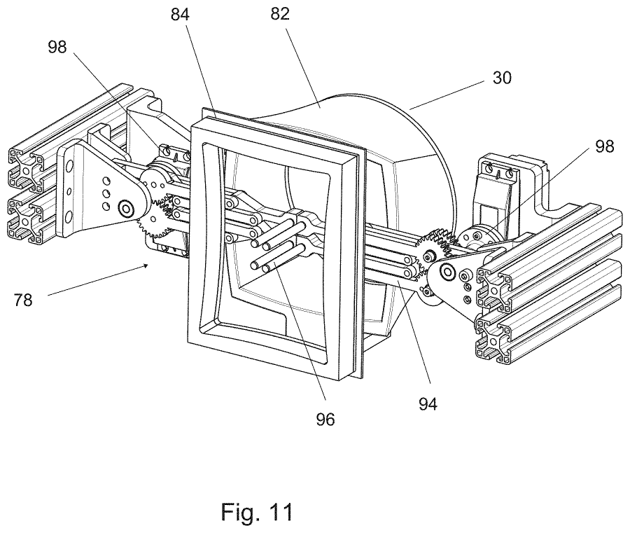

FIG. 11 shows cuff-stretching and fixing device 78, which includes cuff-stretching claw arms 94 (exemplified by articulated cuff stretching arm segments); cuff-stretching pins 96 at the free ends of the claws arms; and cuff-stretching claw motors 98. Cuff-stretching claw arms 94 pass into opposing sides of a gap between gateway 82 and gateway frame 84. In FIG. 11, cuff-stretching pins 96 are in a “closed” or bunched position ready to stretch the cuff.

FIG. 13 shows cuff-stretching pins 96 in a spread (cuff open) position. 20 Activation of cuff opening device 76, specifically activation of cuff-stretching claw motors 98, causes cuff-stretching pins 96 to spread outward away from each other, for example, radially in an x-shaped direction to the corners of frame 84, as illustrated, to thereby stretch and hold the cuff to essentially open the cuff to the inner periphery of frame 84. At this time, glove-picking vacuum pump 62 and suction-plate vacuum pump 90 are deactivated.

The glove is inflated when glove inflation device 80 is activated by way of operation of a cabinet air-extraction fan 102 (FIG. 1) to lower the pressure within cabinet 20. The pressure drop within cabinet 20 causes the glove to inflate, whereby the glove is fully open and the user can insert their hand. Sensor 28 or instruction screen 35, or another appropriate indicator, sensor or screen, may provide the user an indication of when the glove is ready to be worn.

It should be understood that the glove can be donned at the point when cuff opening device 76 partially opens the glove, or at the stage where the a cuff-stretching and fixing device 78 stretches the cuff. However, the most convenient point for donning the glove is when the glove is inflated.

When a user wishes to don a glove, the user can first actuate a dose of sanitizer by placing the hand adjacent sanitizer dispensing sensor 34 supplied from sanitizer compartment 32. After hand sanitizing, the user places their hand in front of glove dispensing sensor 28. The user can choose the desired size (e.g. large, medium, small) using touch pad/sensor 28, and/or the dispenser can read an employee badge (e.g. with a bar or QR code) whereby the user's hand size will be known.

Sensor 28 actuates the process, which activates rotation (if necessary) of carousel 36 to position glove-supply sleeve 42 having a glove of the desired size at the glove picking position. Glove picking and positioning mechanism 56 is then activated to pick up and move the glove opposite frame 84. At this point cuff opening device 76 opens the glove's cuff and dresses the cuff on frame 84. The glove is then inflated by glove inflation device 80 and the glove is ready for donning.

The user then inserts their hand through port 30 and gateway 82 and into the glove to remove the glove's cuff from frame 84. The dispenser may include a sensor to recognize the hand insertion to stop the dispenser activity (i.e., at this point, to turn off air-extraction fan 102).

As a result, the user can don a glove with absolutely no need to touch the outside of the glove, for donning or any other reason. Thus, the automatic donning of the glove is made simple and quick without contamination.

It should be understood that the above description is merely exemplary and various embodiments of the present invention may be devised, mutatis mutandis, and that the features described in the above-described embodiments, and those not described herein, may be used separately or in any suitable combination; and the invention can be devised in accordance with embodiments not necessarily described above.

1. A glove dispenser for dispensing a glove, the dispenser comprising:
a cabinet with a hand-insertion port;
a glove storage container housed within the cabinet and comprising at least one glove bin configured to store a plurality of gloves;
a glove picking and positioning mechanism configured to remove one glove from one of the at least one glove storage bins and to fix the glove in a position accessible via the hand-insertion port; and
a glove-cuff opening device.

2. The dispenser of claim 1, wherein the glove picking and positioning mechanism comprises a glove picking arm with a glove-picking element attached to its free end.

3. The dispenser of claim 2, wherein the glove-picking element has at least one glove-picking vacuum openings operably connected to a glove-picking vacuum pump.

4. The dispenser of claim 1, wherein the glove picking arm is connected to one or more picking-arm movement rods upon which the picking arm can be moved back and forth.

5. The dispenser of claim 1, wherein each of the at least one glove bin is configured so that the gloves therein are arranged in a vertically stacked arrangement.

6. The dispenser of claim 1, wherein the at least one glove storage bin is a plurality of storage bins.

7. The dispenser of claim 1, wherein the plurality of storage bins is arranged radially in the glove storage container in an equidistant petal-like arrangement.

8. The dispenser of claim 1, wherein the glove storage container is configured as a rotatable carousel.

9. The dispenser of claim 8, wherein the glove storage container is associated with a storage bin rotation mechanism configured to rotate the glove storage container.

10. The dispenser of claim 1, further comprising a cuff opening device configured to open the cuff of the glove and comprising a cuff-opening suction plate operably connected to and moveable by a suction-plate movement motor and operably connected to a suction-plate vacuum pump.

11. The dispenser of claim 1, further comprising a cuff-stretching and fixing device configured to stretch the cuff of the positioned glove.

12. The dispenser of claim 11, wherein the cuff stretching and fixing device is configured to stretch the glove's cuff adjacent to the inner periphery of a hand-insertion gateway frame.

13. The dispenser of claim 11, further comprising a gateway frame upon which the glove's cuff is fixable by the cuff-stretching and fixing device.

14. The dispenser of claim 11, wherein the cuff-stretching and fixing device comprises cuff-stretching claw arms cuff-stretching pins at free ends of the claw arms; and cuff-stretching claw motors configured to move claw arms so as to spread and converge the cuff-stretching pins.

15. The dispenser of claim 1, further comprising a glove request sensor or instruction screen configured to allow a user to request one glove of the plurality of gloves.

16. The dispenser of claim 1, further comprising an instruction screen configured to allow a user to indicate a desired glove size.

17. The dispenser of claim 1, further comprising a cabinet air-extraction fan for inflating the glove.

18. The dispenser of claim 1, wherein each glove bin has associated therewith a linear glove-stack pushing device configured to raise a stack of gloves located within each glove bin.

19. The dispenser of claim 1, further comprising a hand sanitizer compartment for housing a hand sanitizer, the dispensing of the sanitizer being actuatable via a sanitizer dispensing sensor.

20. A glove opening mechanism for a glove having a cuff, the mechanism comprising:
a glove picking arm with a glove-picking element attached to its free end, the glove-picking element;
a glove-picking vacuum pump operably attached to the glove-picking element (58); and
a cuff-opening suction plate (86) and operably connected to a suction-plate vacuum pump.

21. The mechanism of claim 20, further including a plurality of glove opening pins configured to enter the glove's cuff and a glove-cuff opening device configured to spread the pins within the cuff.

22. The mechanism of claim 21, wherein the glove-cuff opening device comprises cuff-stretching claw arms; cuff-stretching pins at free ends of the claw arms; and cuff-stretching claw motors operably connected to the claw arms and configured to move claw arms so as to spread and converge the plurality of cuff-stretching pins.

23. The mechanism of claim 20, further including a cabinet air-extraction fan configured to lower the pressure within the cabinet for inflating the glove.
